(19) United States
(12) Patent Application Publication
(10) Pub. No.: US 2015/0366484 A1
(43) Pub. Date: Dec. 24, 2015

(54) METHOD FOR DETERMINING A SPATIALLY RESOLVED DISTRIBUTION OF A MARKER SUBSTANCE

(71) Applicant: SIEMENS AKTIENGESELLSCHAFT, MÜNCHEN (DE)

(72) Inventor: DAVID GRODZKI, ERLANGEN (DE)

(21) Appl. No.: 14/739,019

(22) Filed: Jun. 15, 2015

(30) Foreign Application Priority Data
Jun. 18, 2014 (DE) 102014211695.3

(51) Int. Cl. A61B 5/055

(52) U.S. Cl. CPC A61B 5/055

(57) ABSTRACT

A method for determining a spatially resolved distribution of a marker substance, a marker substance and a use of a marker substance in a quantitative magnetic resonance method is provided. To specify an effective possibility of determining a spatially resolved distribution of a marker substance in an object under examination, the method for determining a spatially resolved distribution of a marker substance, located in an object under examination, includes: acquiring magnetic resonance signals of an examination region of the object under examination by means of a quantitative magnetic resonance method, quantifying a measurement-n-tuple of material parameters with the aid of the acquired magnetic resonance signals, comparing the measurement-n-tuple with a known marker substance-n-tuple of the marker substance, calculating a spatially resolved distribution of the marker substance in the examination region with the aid of the result of the comparison, and providing the spatially resolved distribution of the marker substance.

This application claims priority to DE 102014211695.3, having a filing date of Jun. 18, 2014, the entire contents of which are hereby incorporated by reference.

### FIELD OF TECHNOLOGY

The following relates to a method for determining a spatially resolved distribution of a marker substance, a marker substance, and a use of a marker substance in a quantitative magnetic resonance method.

### BACKGROUND

In a magnetic resonance device, also known as a magnetic resonance tomography system, the body of the subject to be examined, particularly that of a patient, is typically exposed to a relatively strong magnetic field of, for example, 1.5 or 3 or 7 Tesla, with the aid of a main magnet. In addition, gradient pulses are played out with the aid of a gradient coil unit. By means of a high frequency antenna unit, using suitable antenna devices, high frequency pulses, particularly excitation pulses, are transmitted, which leads to the nuclear spins of particular atoms being excited into resonance by these high frequency pulses are tilted through a defined flip angle relative to the magnetic field lines of the main magnetic field. On relaxation of the nuclear spin, high frequency signals known as “magnetic resonance signals” are emitted and are received by suitable high frequency antennas and then further processed. From the raw data thereby acquired, the desired image data can ultimately be reconstructed.

Marker substances can be used in the magnetic resonance imaging. These marker substances can be visualized in magnetic resonance images recorded using the magnetic resonance device. Substances, for instance drugs or radioactive applicators for radiotherapy, can be marked for instance using the marker substance so that a distribution of the substance in a body of an object under examination can be displayed using magnetic resonance imaging. A statement relating to a metabolism of the object under examination can also be obtained directly using a visualization of a distribution of the marker substance if the marker substance is embodied as a contrast agent.

### SUMMARY

An aspect relates to specifying an effective possibility of determining a spatially resolved distribution of a marker substance in an object under examination.

Embodiments of the invention are based on a method for determining a spatially resolved distribution of a marker substance, which is disposed in an object under examination, including the following method steps:
acquiring magnetic resonance signals of an examination region of the object under examination by means of a quantitative magnetic resonance method,
quantifying a measurement-n-tuple of material parameters with the aid of the acquired magnetic resonance signals,
comparing the measurement-n-tuple with a known marker substance-n-tuple of the marker substance,
calculating a spatially resolved distribution of the marker substance in the examination region with the aid of the result of the comparison and
providing the spatially resolved distribution of the marker substance.

The object under examination can be a patient, a training person or a phantom. The marker substance is disposed in the examination region in particular before the start of the acquisition of the magnetic resonance signals. Magnetic resonance signals based on the marker substance are inter alia also acquired. The marker substance can also be embodied to mark a substance. To this end, the marker substance can interact with the substance. The marker substance can be embodied as a contrast agent for the magnetic resonance imaging. The marker substance advantageously accumulates in a tissue of the object under examination. The marker substance is in this case advantageously embodied statically in the object under examination, in other words it moves as little as possible in the tissue during the acquisition of the magnetic resonance signals. The marker substance can be embodied for instance in a diamagnetic manner.

A quantitative magnetic resonance method, which is used to acquire the magnetic resonance signals, is used in particular to determine quantitative material parameters. Here a quantitative magnetic resonance method advantageously allows for a quantification of the material parameter, which is independent for instance of measurement conditions or of a type of magnetic resonance device. The material parameters can thus be reproducibly reconstructed with the aid of magnetic resonance signals acquired in a quantitative magnetic resonance method. A quantitative magnetic resonance image reconstructed from a quantitative magnetic resonance method can thus advantageously contain information relating to the absolute physical variables. A value of an image pixel of such a quantitative magnetic resonance image is thus advantageously directly related to a physical measured value. The value of an image pixel may comprise in particular a physical unit. The value of the image pixel of the quantitative magnetic resonance image is advantageously independent here of measurement parameters used during the recording of the magnetic resonance image data, adjustment settings, types of magnetic resonance devices, recording coils used etc. Therefore, recorded magnetic resonance images can be advantageously compared directly with one another by means of different quantitative magnetic resonance methods, possibly under different measurement conditions. By contrast the image information is based on magnetic resonance images obtained from non-quantitative, in particular qualitative methods, for instance magnetic resonance images with a T2 weighting, typically only on a signal comparison within the magnetic resonance image. Such non-quantitative magnetic resonance images therefore typically only supply a qualitative contrast between different substances. The values of the image pixels of non-quantitative magnetic resonance images can differ from examination to examination even with the same selection of parameters. The signal intensities determined in non-quantitative methods typically do not directly map a physical value. A statement relating to the physical properties which underlie the signal intensities can typically only be made with an evaluation of different signal intensities of a non-quantitative magnetic resonance image relative to one another.

Material parameters are in particular quantified by means of the magnetic resonance signals acquired in the quantitative magnetic resonance method. The quantification of the material parameters is performed in particular in a spatially resolved manner. Therefore a spatially resolved distribution of the material parameters is in particular quantified. The material parameters advantageously characterize a physical property of the substance, for instance the marker substance, from which the magnetic resonance signals are acquired. In particular, the material parameters can quantify a reaction of the substance to a high frequency excitation.

The quantified material parameters are in particular grouped together into the measurement-n-tuple. A first entry of the measurement-n-tuple can thus represent a first quantified material parameter, a second entry of the measurement-n-tuple can represent a second quantified material parameter etc. It is also conceivable for only one material parameter to be quantified, so that a measurement-1-tuple is quantified. However, it is advantageous if a number of material parameters are quantified. The number of material parameters in the measurement-n-tuple is thus advantageously greater than 1. The measurement-n-tuple can be a measurement-3-tuple for instance. The measurement-n-tuple includes in particular a maximum of 30 material parameters, preferably a maximum of 20 material parameters, advantageously a maximum of ten material parameters, at most advantageously a maximum of five material parameters. A measurement-n-tuple of the material parameters is advantageously quantified for each pixel of the examination region. The measurement-n-tuple is in particular structured in a similar manner to the marker substance-n-tuple and/or the tissue-n-tuple.

A selection of possible material parameters, which may form the measurement-n-tuple, is: a T1 relaxation time, a T2 relaxation time, a diffusion value (for instance an apparent diffusion coefficient ADC), a magnetization torque, a proton density, a resonance frequency, a concentration of a substance etc. Other further material parameters which are useful to the person skilled in the art are naturally also conceivable. Any combination of the measurement-n-tuple can form from the cited material parameters. The material parameters can be determined here directly by means of the quantitative magnetic resonance method. Here the material parameters can be pixel values of the magnetic resonance images acquired by means of the quantitative magnetic resonance method. The quantitative magnetic resonance method thus allows for a direct characterization of the material parameter.

The known marker substance-n-tuple includes in particular the material parameters of the marker substance. The marker substance-n-tuple here is in particular stored in a database. It may have been determined beforehand by means of a measurement or on account of a priori specialist knowledge relating to the material properties of the marker substance. It is possible to determine on the basis of the comparison of the measurement-n-tuple with the marker substance-n-tuple the degree to which the measurement-n-tuple corresponds with the marker substance-n-tuple. In such cases the material parameters of the measurement-n-tuple can be compared individually or in combination with the material parameters of the marker substance-n-tuple.

The spatially resolved distribution of the marker substance in the examination region includes in particular an item of information relating to the location in which the marker substance is found in the examination region. A binary assignment can take place here, in other words a distinction is made as to whether or not the marker substance is located at a location. This binary assignment can take place with the aid of a threshold value of a similarity of the marker substance-n-tuple and of the measurement-n-tuple defined during the comparison. It is also conceivable for the spatially resolved distribution to be graded discretely, in particular with more than two levels, or embodied continuously. The spatially resolved distribution of the marker substance can for instance include similarity values between the marker substance-n-tuple and the measurement-n-tuple. The similarity values can be determined when comparing the measurement-n-tuple with the marker substance-n-tuple.

The provision of the spatially resolved distribution can include a display of the spatially resolved distribution for a user, for instance on a monitor. Alternatively or in addition, the provision of the spatially resolved distribution can include a storing of the spatially resolved distribution, for instance on a database.

The proposed procedure offers an effective possibility of determining the spatially resolved distribution of the marker substance. A reproducible determination of the distribution of the marker substance is also possible, so that with a repetition of the measurement, a temporal change in the spatially resolved distribution of the marker substance can be shown in a particularly meaningful manner. The marker substance can also be particularly advantageously selected for the quantitative magnetic resonance method. The material parameters of the marker substance can thus be adjusted to the selected quantitative magnetic resonance measurement, as a result of which an advantageous visualization of the marker substance is possible.

One embodiment provides that the quantitative magnetic resonance method is a magnetic resonance fingerprinting method. A possible magnetic resonance fingerprinting method is for instance known from the publication Ma et al., “Magnetic Resonance Fingerprinting”, Nature 495, 187-192 (Mar. 14, 2013). With a magnetic resonance fingerprinting method, a number of magnetic resonance images of the examination region are typically acquired, wherein different recording parameters are set for the acquisition of the various magnetic resonance images. The recording parameters can be varied here in a pseudo randomized manner. Possible recording parameters, which are changed during the acquisition of the number of magnetic resonance images, are for instance an echo time, an embodiment and/or number of high frequency pulses, a development and/or number of gradient pulses, a diffusion encoding etc. The number of magnetic resonance images can be acquired here during a number of repetition times, wherein a magnetic resonance image of the several magnetic resonance images can be acquired in each case during a repetition time of the number of repetition times respectively. A spatially dependent magnetic resonance signal curve is then typically generated by way of the number of magnetic resonance images. This magnetic resonance signal curve is then typically compared with a number of database signal curves stored in a database in a signal comparison. A different database-n-tuple of material parameters is advantageously assigned here to the various database signal curves in each case. The database signal curve then represents in each case the signal curve to be expected from the magnetic resonance fingerprinting method, if a sample the material properties of which correspond to those of the associated database-n-tuple, is examined. The database signal curves can be determined and/or simulated for instance in a calibration measurement. The magnetic resonance fingerprinting method then typically provides that a database signal curve of the number of database signal curves is assigned to the generated magnetic resonance signal curve with the aid of the result of the signal comparison. The database-n-tuple associated with the assigned database signal curve can then be set as a measurement-n-tuple. The magnetic resonance fingerprinting method thus allows for a particularly advantageous quantification of the measurement-n-tuple. All material parameters of the measurement-n-tuple can in particular be determined at the same time by means of a magnetic resonance fingerprinting method. Only the acquisition of an individual magnetic resonance signal curve is required for a voxel of the examination region, in order to determine all material parameters of the measurement-n-tuple by means of the magnetic resonance fingerprinting method for the voxel.

One embodiment provides that the measurement-n-tuple of the material parameters includes at least one of the following material parameters: a T1 relaxation time, a T2 relaxation time, a resonance frequency. These material parameters cited here represent particularly advantageous material parameters for the measurement-n-tuple. The measurement-n-tuple may also be for instance a measurement-3-tuple and can be formed by the T1 relaxation time, the T2 relaxation time and the resonance frequency.

One embodiment provides that the marker substance is adjusted in such a way to a tissue of the object under examination located in the examination region that the marker substance differs from a known tissue-n-tuple of the tissue by at least 20 percent in at least one material parameter of the marker substance n-tuple. The marker substance differs in particular in the at least one material parameter of the marker substance-n-tuple from the known tissue-n-tuple by at least 40 percent, preferably by at least 60 percent, advantageously by at least 80 percent, at most advantageously by at least 100 percent. The tissue-n-tuple may have been determined beforehand by means of a measurement or on account of a priori specialist knowledge relating to the material properties of the tissue. In this way the marker substance is adjusted particularly advantageously to the tissue for the visualization by means of the quantitative magnetic resonance method. A particularly good limitation of the marker substance by the tissue is namely possible. The marker substance can thus be visualized particularly easily. The adjustment of the marker substance to the tissue may include a suitable selection and/or change to the marker substance. The marker substance can contain protons or other magnetic resonance-visible nuclei, which have material parameters which differ sufficiently from the material parameters of the tissue. An addition of magnesium or iron oxide to the marker substance is conceivable for instance so that a T1 relaxation time and/or a T2 relaxation time of the marker substance is set and can advantageously be adjusted to the tissue.

One embodiment provides that the tissue located in the examination region is determined with the aid of a localization of the examination region in a body of the object under examination. Different tissue-n-tuples, which belong to different tissue types, can be stored in a database. Depending on the localization of the examination region in the body of the object under examination, the suitable tissue-n-tuple of the various tissue-n-tuples can then be loaded from the database for comparison with the marker substance-n-tuple. Alternatively or in addition, the suitable tissue type can also be determined with the aid of an item of information, in which tissue type the marker substance typically accumulates. If various tissue types are located in the examination region, various tissue-n-tuples can be loaded from the database for comparison with the marker substance-n-tuple. The tissue located in the examination region can thus be determined particularly easily and a comparison basis can be created for the measurement-n-tuple.

One embodiment provides that the measurement-n-tuple is compared with the marker substance-n-tuple and the tissue-n-tuple, and an assignment of the measurement-n-tuple to the marker substance-n-tuple or the tissue-n-tuple takes place with the aid of the result of the comparison. By comparison with the marker substance-n-tuple, the comparison of the measurement-n-tuple with the tissue-n-tuple also offers an advantageous possibility of determining whether the marker substance is located at a specific location. The tissue-n-tuple thus represents a reference basis which can be used to estimate the comparison of the measurement-n-tuple with the marker substance-n-tuple. If the measurement-n-tuple is assigned to the marker substance-n-tuple, it can be determined that the marker substance is located at the corresponding point in the spatially resolved distribution. If the measurement-n-tuple is assigned to the tissue-n-tuple, it can be determined that the marker substance is not located at the corresponding point in the spatially resolved distribution.

One embodiment provides that the assignment of the measurement-n-tuple to the marker substance-n-tuple or to the tissue-n-tuple takes place according to the criterion as to whether the material parameters of the measurement-n-tuple are more similar to the material parameters of the marker substance-n-tuple or to the material parameters of the tissue-n-tuple. A first similarity measure, which describes the similarity of the measurement-n-tuple and the marker substance-n-tuple and a second similarity measure which describes the similarity of the measurement-n-tuple and the tissue-n-tuple can be determined for instance. If the first similarly measure is greater than the second similarity measure, the assignment of the measurement-n-tuple to the marker substance-n-tuple can thus take place. If the second similarity measure is greater than the first similarity measure, the assignment of the measurement-n-tuple to the tissue-n-tuple can thus take place. Here the similarity measure can describe the degree of the deviation, for instance in percentage, of the two n-tuples. It can thus be particularly easily determined, whether the measurement-n-tuple is to be assigned to the marker substance-n-tuple or to the tissue-n-tuple.

One embodiment provides that the marker substance changes temporally in the at least one material parameter of the marker substance-n-tuple, wherein a temporal state of the change in the marker substance is determined with the aid of the quantification of the measurement-n-tuple. The at least one material parameter of the marker substance changes in particular temporally, while the marker substance is located in the object under examination. The marker substance is embodied for instance such that it has a membrane, through which water can enter or escape over the course of time. The water movement can take place for instance in conjunction with a pharmaceutical. Over the course of time, a concentration of magnetic resonance-visible nuclei can thus change within the membrane, and the marker substance can thus change temporally in at least one material parameter. An item of information relating to a temporal change in the at least one material parameter can be stored in a database. If a presence of the marker substance has been determined at a location in the examination region, a temporal state of the change in the marker substance can be determined with the aid of the measurement-n-tuple and the information relating to the temporal change in the at least one material parameter. A comparison of the measurement-n-tuple with various marker substance-n-tuples can also take place, which are assigned to different temporal states of the marker substance. The determination of the temporal state of the change in the marker substance can provide the person skilled in the art for instance with an item of information as to which quantity of pharmaceutical has already escaped from the marker substance. Further applications which appear useful to the person skilled in the art are also conceivable.

Furthermore embodiments of the invention are based on a magnetic resonance device with a signal acquisition unit, a computing unit and a provisioning unit, wherein the magnetic resonance device is embodied to execute an inventive method.

The magnetic resonance device is thus embodied so as to execute a method for determining a spatially resolved distribution of a marker substance, which is located in an object under examination. The signal acquisition unit is embodied to acquire magnetic resonance signals of an examination region of the object under examination by means of a quantitative magnetic resonance method. The computing unit, in particular a quantification unit of the computing unit, is embodied to quantify a measurement-n-tuple of material parameters with the aid of the acquired magnetic resonance signals. The computing unit, in particular a comparison unit of the computing unit, is embodied so as to compare the measurement-n-tuple with a known marker substance-n-tuple of the marker substance. The computing unit, in particular a calculation unit of the computing unit, is embodied to calculate a spatially resolved distribution of the marker substance in the examination region with the aid of the result of the comparison. The provisioning unit is embodied to provide the spatially resolved distribution of the marker substance.

According to an embodiment of the magnetic resonance device, the signal acquisition unit is embodied such that the quantitative magnetic resonance method is a magnetic resonance fingerprinting method.

According to an embodiment of the magnetic resonance device, the signal acquisition unit and the computing unit are embodied such that the measurement-n-tuple of the material parameter includes at least one of the following material parameters; a T1 relaxation time, a T2 relaxation time, a resonance frequency.

According to one embodiment of the magnetic resonance device, the marker substance is adjusted in such a way to a tissue of the object under examination located in the examination region that the marker substance differs from a known tissue-n-tuple of the tissue by at least 20 percent in at least one material parameter of the marker substance n-tuple.

According to an embodiment of the magnetic resonance device, the computing unit is embodied such that the tissue located in the examination region is determined with the aid of a localization of the examination region in a body of the object under examination.

According to an embodiment of the magnetic resonance device, the computing unit is embodied such that the measurement-n-tuple is compared with the marker substance-n-tuple and the tissue-n-tuple, and an assignment of the measurement-n-tuple to the marker substance-n-tuple or the tissue-n-tuple takes place with the aid of the result of the comparison.

According to an embodiment of the magnetic resonance device, the computing unit is embodied such that the assignment of the measurement-n-tuple to the marker substance-n-tuple or to the tissue-n-tuple takes place according to the criterion as to whether the material parameters of the measurement-n-tuple are more similar to the material parameters or the marker substance-n-tuple or to the material parameters of the tissue-n-tuple.

According to an embodiment of the magnetic resonance device, the marker substance changes temporally in the at least one material parameter of the marker substance-n-tuple, and the computing unit is embodied so as to determine a temporal state of the change in the marker substance with the aid of the quantification of the measurement-n-tuple.

Furthermore, embodiments of the invention is based on a use of a marker substance in a quantitative magnetic resonance method for mapping a distribution of the marker substance in a tissue of an object under examination, wherein the marker substance is provided such that it has material parameters which are described in a marker substance-n-tuple and that the marker substance is adjusted to the tissue such that the marker substance differs from a known tissue-n-tuple of the tissue by at least 20 percent in at least one material parameter of the marker substance-n-tuple. A particularly suitable marker substance can accordingly be used in the quantitative magnetic resonance method.

The advantages of the inventive use of the marker substance and of the inventive magnetic resonance device essentially correspond to the advantages of the inventive method, which are explained above in detail. Features, advantages or alternative embodiments mentioned herein are also to be applied to the other claimed subject matter and vice versa. In other words, the present claims can also be further developed with the features disclosed or claimed in conjunction with a method. The corresponding functional features of the method are configured here by means of suitable modules as contained herein, in particular by means of hardware modules.

### BRIEF DESCRIPTION

Some of the embodiments will be described in detail, with reference to the following figures, wherein like designations denote like members, wherein:

FIG. 1 shows an embodiment of a magnetic resonance device in a schematic representation;

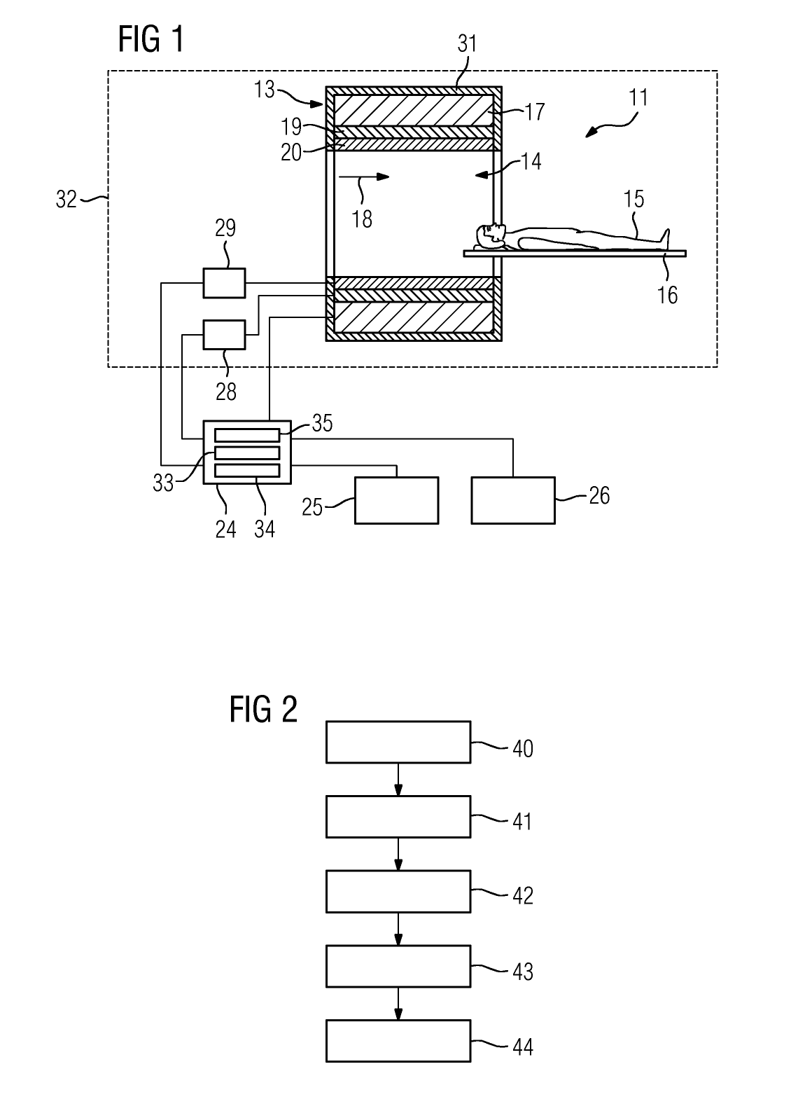

FIG. 2 shows a flow chart of a first embodiment of a method; and

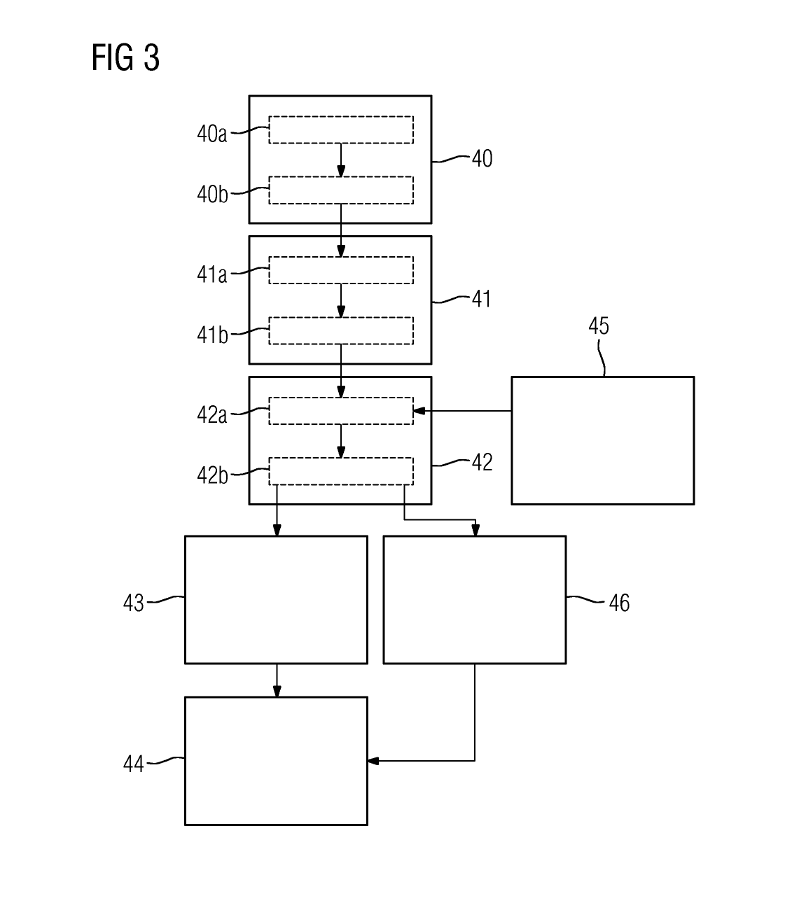

FIG. 3 shows a flow chart of a second embodiment of a method.

### DETAILED DESCRIPTION

FIG. 1 schematically illustrates an inventive magnetic resonance device 11. The magnetic resonance device 11 comprises a detector unit consisting of a magnet unit 13 with a main magnet 17 for generating a strong and, particularly constant, main magnetic field 18. The magnetic resonance device 11 additionally has a patient receiving zone 14 in the shape of a cylinder for receiving an object under examination 15, in the present case a patient 15, the patient receiving zone 14 being cylindrically surrounded by the magnet unit 13 in a circumferential direction. The patient 15 can be pushed by means of a patient support apparatus 16 of the magnetic resonance device 11 into the patient receiving zone 14. To this end the patient positioning apparatus 16 has a couch, which is disposed in a movable manner within the magnetic resonance device 11. The magnet unit 13 is screened toward the outside by means of a housing covering 31 of the magnetic resonance device.

The magnet unit 13 also has a gradient coil unit 19 for generating magnetic field gradients that are used for position encoding during imaging. The gradient coil unit 19 is controlled by means of a gradient control unit 28. Furthermore, the magnet unit 13 has a high frequency antenna unit 20 which, in the case shown is configured as a body coil firmly integrated into the magnetic resonance device 10, and a high frequency antenna control unit 29 for excitation of a polarization which is created in the main magnetic field 18 generated by the main magnet 17. The high-frequency antenna unit 20 is controlled by the high-frequency antenna control unit 29 and radiates high-frequency magnetic resonance sequences into an examination space that is substantially formed by the patient receiving zone 14. The high-frequency antenna unit 20 is further designed to receive magnetic resonance signals, in particular from the patient 15.

For the purpose of controlling the main magnet 17, the gradient control unit 28 and the high-frequency antenna control unit 29, the magnetic resonance device 11 has a computing unit 24. The computing unit 24 is used for central control of the magnetic resonance device 11, such as performing a predetermined imaging gradient echo sequence for example. Control information such as, for example, imaging parameters and reconstructed magnetic resonance images can be provided for a user on a provisioning unit 25, in the present case a display unit 25, of the magnetic resonance device 11. Furthermore, the magnetic resonance device 11 has an input unit 26, by means of which information and/or parameters can be entered by a user during a measurement procedure. The computing unit 24 can comprise the gradient control unit 28 and/or the high frequency antenna control unit 29 and/or the display unit 25 and/or the input unit 26.

In the case shown, the computing unit 24 includes a quantification unit 33, a comparison unit 34 and a calculation unit 35.

The magnetic resonance device 11 further includes a signal acquisition unit 32. The signal acquisition unit 32 is formed in the present case by the magnet unit 13 together with the high frequency antenna control unit 29 and the gradient control unit 28. The magnetic resonance device 11 is thus designed, together with the signal acquisition unit 32, the computing unit 24 and the provisioning unit 25, to perform an inventive method.

The magnetic resonance device 11 disclosed can naturally comprise further components which magnetic resonance devices 11 typically have. A general method of functioning of a magnetic resonance device 11 is also known to the person skilled in the art, so that a detailed description of the further components is not included.

FIG. 2 shows a flow chart of a first embodiment of an inventive method for determining a spatially resolved distribution of a marker substance which is located in an object under examination 15.

In a first method step 40, the signal acquisition unit 32 of the magnetic resonance device 11 acquires magnetic resonance signals of an examination region of the object under examination 15 by means of a quantitative magnetic resonance method. In a further method step 41, a quantification of a measurement-n-tuple of material parameters takes place with the aid of the acquired magnetic resonance signals by means of the quantification unit 33 of the computing unit 24. In a further method step 42, a comparison of the measurement-n-tuple with a known marker substance-n-tuple of the marker substance takes place by means of the comparison unit 34 of the computing unit 24. In a further method step 43, a calculation of a spatially resolved distribution of the marker substance in the examination region takes place with the aid of the result of the comparison by means of the calculation unit 35 of the computing unit 24. In a further method step 44, the spatially resolved distribution of the marker substance is provided by means of the provisioning unit 25 of the magnetic resonance device 11.

FIG. 3 shows a flow chart of a second embodiment of an inventive method for determining a spatially resolved distribution of a marker substance which is located in an object under examination 15.

The following description is essentially restricted to the differences from the exemplary embodiment in FIG. 2 wherein, with regard to method steps which remain the same, reference is made to the description of the exemplary embodiment in FIG. 2. In principle, the same method steps are essentially identified with the same reference signs.

The second embodiment of the method according to the invention shown in FIG. 3 essentially comprises the method steps 40, 41, 42, 43 of the first embodiment of the method according to the invention as shown in FIG. 2. The second embodiment of the method according to the invention shown in FIG. 3 additionally comprises further method steps and sub-steps. Also conceivable is an alternative method sequence to that of FIG. 3 which has only part of the additional method steps and/or sub-steps represented in FIG. 2. Naturally, an alternative method sequence to that of FIG. 3 can also have additional method steps and/or sub-steps.

The magnetic resonance method, which is used in the first method step 40 to acquire the magnetic resonance signals, is a magnetic resonance fingerprinting method. Accordingly, the quantification of the measurement-n-tuple of the material parameter is also adjusted in the further method step 41 to the magnetic resonance fingerprinting method. The first method step 40 thus includes a first sub-step 40a, in which a number of magnetic resonance images are acquired by means of the magnetic resonance fingerprinting method, in other words using different recording parameters. The first method step 40 further includes a second sub-step 40b, in which a magnetic resonance signal course is generated over the number of magnetic resonance images. The further method step 41 includes a first sub-step 41a, in which the magnetic resonance signal course is compared with various database signal courses, which are assigned to different database-n-tuples of material parameters, in a signal comparison. The further method step 41 further includes a second sub-step 41b, in which a certain database signal course is assigned to the measured magnetic resonance signal course with the aid of the result of the signal comparison. The database-n-tuple associated with the certain database signal course is then set as a measurement-n-tuple.

The measurement-n-tuple of the material parameter in the exemplary embodiment shown in FIG. 3 forms a measurement-3-tuple by way of example. Three material parameters in the further method step 41 are accordingly quantified by the quantification unit 33 of the computing unit 24. The measurement-3-tuple includes by way of example a T1 relaxation time, a T2 relaxation time and a resonance frequency as material parameters. A deviating number of material parameters in the measurement-n-tuple is naturally also conceivable. The measurement-n-tuple can also include other material parameters which appear useful to the person skilled in the art.

In a further method step 45, a tissue located in the examination region is determined by means of the computing unit 24 with the aid of a localization of the examination region in a body of the object under examination 15. The marker substance is adjusted in such a way to the tissue of the object under examination which is located in the examination region such that the marker substance differs from a known tissue-n-tuple of the tissue by at least 20 percent in at least one material parameter of the marker substance n-tuple. For instance, the marker substance has a resonance frequency which is 50 percent greater than the resonance frequency of the tissue. In a further method step 42, the comparison unit 34 of the computing unit can therefore distinguish the marker substance from the tissue, in which the marker substance is located, particularly easily. Therefore in a first sub-step 42a of the further method step 42 by means of the comparison unit 34, the measurement-n-tuple is compared with the marker substance-n-tuple and the tissue-n-tuple. An assignment of the measurement-n-tuple to the marker substance-n-tuple or to the tissue-n-tuple by means of the comparison unit 34 takes place with the aid of the result of the comparison in a second sub-step 42b of the further method step 42. The assignment of the measurement-n-tuple to the marker substance-n-tuple or to the tissue-n-tuple takes place for instance according to the criterion as to whether the material parameters of the measurement-n-tuple are more similar to the material parameters of the marker substance-n-tuple or to the material parameters of the tissue-n-tuple.

In the case shown in FIG. 3, the procedure includes a further method step 45, in which a temporal state of the change in the marker substance is determined with the aid of the quantification of the measurement-n-tuple. This is possible since the marker substance changes temporally in at least one material parameter of the marker substance-n-tuple. An item of information relating to the temporal state of the change in the marker substance can be provided in the further method step 44 for a user by means of the provisioning unit 25.

Although the invention has been illustrated and described in greater detail on the basis of the preferred exemplary embodiments, the invention is not limited by the disclosed examples and other variations can be derived herefrom by the person skilled in the art without departing from the scope of protection of the invention.

1. A method for determining a spatially resolved distribution of a marker substance, which is disposed in an object under examination, the method comprising:
acquiring magnetic resonance signals of an examination region of the object under examination by means of a quantitative magnetic resonance method;
quantifying a measurement-n-tuple of material parameters with the aid of the acquired magnetic resonance signals;
comparing the measurement-n-tuple with a known marker substance-n-tuple of the marker substance;
calculating a spatially resolved distribution of the marker substance in the examination region with the aid of the result of the comparison; and
providing the spatially resolved distribution of the marker substance.

2. The method as claimed in claim 1, wherein the quantitative magnetic resonance method is a magnetic resonance fingerprinting method.

3. The method as claimed in claim 1, wherein the measurement-n-tuple of the material parameter includes at least one of the following material parameters: a T1 relaxation time, a T2 relaxation time, and a resonance frequency.

4. The method as claimed in claim 1, wherein the marker substance is adjusted in such a way to a tissue of the object under examination located in the examination region that the marker substance differs from a known tissue-n-tuple of the tissue by at least 20 percent in at least one material parameter of the marker substance n-tuple.

5. The method as claimed in claim 4, wherein the tissue located in the examination region is determined with the aid of a localization of the examination region in a body of the object under examination.

6. The method as claimed in one of claim 4, wherein the measurement-n-tuple is compared with the marker substance-n-tuple and the tissue-n-tuple, and an assignment of the measurement-n-tuple to the marker substance-n-tuple or the tissue-n-tuple takes place with the aid of the result of the comparison.

7. The method as claimed in claim 6, wherein the assignment of the measurement-n-tuple to the marker substance-n-tuple or to the tissue-n-tuple takes place according to the criterion as to whether the material parameters of the measurement-n-tuple are more similar to the material parameters of the marker substance-n-tuple or to the material parameters of the tissue-n-tuple.

8. The method as claimed in claim 1, wherein the marker substance changes temporally in the at least one material parameter of the marker substance-n-tuple, wherein a temporal state of the change in the marker substance is determined with the aid of the quantification of the measurement-n-tuple.

9. A magnetic resonance device having a signal acquisition unit, a computing unit and a provisioning unit, wherein the magnetic resonance device executes the method as claimed in claim 1.

10. A marker substance in a quantitative magnetic resonance method for mapping a distribution of the marker substance in a tissue of an object under examination, wherein the marker substance is provided such that it has material parameters which are described in a marker substance-n-tuple and that the marker substance is adjusted to the tissue such that the marker substance differs from a known tissue-n-tuple of the tissue by at least 20 percent in at least one material parameter of the marker substance-n-tuple.
